(19) United States
(12) Patent Application Publication
(10) Pub. No.: US 2013/0305452 A1
(43) Pub. Date: Nov. 21, 2013

(54) GANTRY-DIAGNOSTIC BED SYSTEM AND FOLDING METHOD THEREOF

(71) Applicants: Toshiba Medical Systems Corporation, Otawara-shi (JP); KABUSHIKI KAISHA TOSHIBA, Tokyo (JP)

(72) Inventors: Pei ZHONGQIANG, Dalian (CN); Zhang Junhui, Dalian (CN); Pang Xuebo, Dalian (CN)

(73) Assignees: Toshiba Medical Systems Corporation, Otawara-shi (JP); KABUSHIKI KAISHA TOSHIBA, Tokyo (JP)

(21) Appl. No.: 13/896,811

(22) Filed: May 17, 2013

(30) Foreign Application Priority Data
May 17, 2012 (CN) 201210154418.9

(51) Int. Cl. A61B 6/04

(52) U.S. Cl. CPC A61B 6/0407
USPC 5/601

(57) ABSTRACT

According to one embodiment, a gantry-diagnostic bed system is provided. The gantry-diagnostic bed system comprises a gantry for supporting a medical device, a diagnostic bed designed as being integral with the gantry and for carrying an object to be inspected, and a diagnostic bed adjusting mechanism for adjusting the position of the diagnostic bed relative to the gantry such that the diagnostic bed can be folded with respect to the gantry.

### CROSS-REFERENCE TO RELATED APPLICATIONS

This application is based upon and claims the benefit of priority from Chinese Patent Application No. 201210154418.9, filed May 17, 2012, the entire contents of which are incorporated herein by reference.

### FIELD

Embodiments described herein relate generally to an integral gantry-diagnostic bed system which is used in medical apparatuses such as CT apparatuses and, more particularly, relates to the whole construction of the system and a folding method thereof.

### BACKGROUND

With the continuous development of medical technologies, CT (computed tomography) apparatuses have been employed widely. Generally, a gantry, in which action components of the CT apparatus are arranged and which is used as a frame carrying the whole CT apparatus, and a diagnostic bed which supports an object to be inspected are separated from each other and installed adjacent each other.

For example, after a hospital purchases a CT apparatus, a gantry and a diagnostic bed of the apparatus are individually packed and transported during the apparatus is conveyed to the hospital. When they arrive at a desired location, they will be assembled together. During assembling, technicians must adjust the positional relationship between the diagnostic bed and the gantry and then fix them. After being assembled together, the gantry and the diagnostic bed are completely fixed. That is, during the CT apparatus is operated, it is immovable if no maintenance is required.

However, because the gantry and the diagnostic bed of the CT apparatus are independent from each other, they have to be individually packed and transported if the CT apparatus is conveyed. Therefore, long time is required to pack them and two independent package boxes are required, which is costly. Further, both the gantry and the diagnostic bed are bulky. Thus after they are unpacked at an installing location, it takes long time to move the gantry and the diagnostic bed so as to adjust the positional relationship between them. It also takes maintainers and doctors long time to assemble them. Therefore, it is not easier to assemble them.

Reference document 1 (JP 4-343837) discloses a method of packing and moving a gantry and a diagnostic bed. In this reference document 1, the positional relationship and the angle between the gantry and the diagnostic bed are reasonably set such that, when packing, they are compactly packed together. Therefore, space is saved and it facilitates deploying the gantry and the diagnostic bed.

However, the reference document 1 is mainly aimed at designing the package. Although, during packing, the positions of the gantry and the diagnostic bed are limited by a plurality walls of the package, the gantry and the diagnostic bed of the reference document 1 are the same as those of the prior art. That is, the gantry and the diagnostic bed are still installed independently from each other. Although deploying them is facilitated, it is still difficult to adjust the positions of the deployed gantry and diagnostic bed. Further, some problems also exist, such as single package being excessive bulky and space being insufficiently utilized.

Further, after the gantry and the diagnostic bed of the prior art are assembled together, they must be completely immovable unless maintenance is required. Even when the CT apparatus is not operated, it can only be remained in its current assembled state. This leads to a problem that the space for placing the apparatus is wasted. Further, when the CT apparatus is moved as re-building the hospital, it is required to separately move the gantry and the diagnostic bed. It is very inconvenient for the maintainers and doctors.

The above problem will be solved by the present embodiment. It is an objective of the present embodiment to provide an integral gantry-diagnostic bed system, by which its gantry and diagnostic bed can be deployed or retracted conveniently and thus can be transported easily, and space is saved.

### BRIEF DESCRIPTION OF THE DRAWINGS

FIG. 1 is a configuration view showing a gantry-diagnostic bed system according to a first embodiment of the present embodiment, which system is in an unfolding state;

FIG. 2 is a configuration view showing the gantry-diagnostic bed system according to the first embodiment before being folded;

FIG. 3 is a configuration view showing the gantry-diagnostic bed system according to the first embodiment during being folded;

FIG. 4 is another configuration view showing the gantry-diagnostic bed system according to the first embodiment during being folded;

FIG. 5 is a configuration view showing the gantry-diagnostic bed system according to the first embodiment, after being folded, in a compact state;

FIG. 6 is a configuration view showing a gantry-diagnostic bed system according to a second embodiment of the present invention, which system is in an unfolding state;

FIG. 7 is a configuration view showing the gantry-diagnostic bed system according to the second embodiment during being folded;

FIG. 8 is a top view showing the gantry-diagnostic bed system according to the second embodiment in the unfolding state;

FIG. 9 is a top view showing the gantry-diagnostic bed system according to the second embodiment in a folding state;

FIG. 10 is a configuration view showing a gantry-diagnostic bed system according to a third embodiment of the present invention in an unfolding state;

FIG. 11 is a configuration view showing the gantry-diagnostic bed system according to the third embodiment during being folded;

FIG. 12 is a configuration view showing the gantry-diagnostic bed system according to the third embodiment during being folded; and

FIG. 13 is a configuration view showing the gantry-diagnostic bed system according to the third embodiment, after being folded, in a compact state.

### DETAILED DESCRIPTION

Preferred embodiments of the present invention will be explained with respect to the accompanying drawings.

In general, according to one embodiment, a gantry-diagnostic bed system is provided. The gantry-diagnostic bed system comprises a gantry for supporting a medical device, a diagnostic bed designed as being integral with the gantry and for carrying an object to be inspected, and a diagnostic bed adjusting mechanism for adjusting the position of the diagnostic bed relative to the gantry such that the diagnostic bed can be folded with respect to the gantry.

### First Embodiment

The idea of the present embodiment is that, because “the gantry and the diagnostic bed are integral and relatively foldable”, the gantry and the diagnostic bed are designed as an integral configuration and the diagnostic bed is foldable relative to the gantry. When the system is normally operated, the gantry and the diagnostic are deployed such that the diagnostic bed can be raised up or lowered down by an actuating mechanism or a supporting member in a known manner. When folding is achieved, the gantry and the diagnostic bed can be relatively folded using the actuating mechanism or an actuating member coupled with them such that they can be transported conveniently and space is saved. In addition, according to the technical solutions of the present embodiment, the gantry and the diagnostic bed are designed in an integral manner such that they can be relatively folded, packed or deployed. Therefore, it facilitates transporting and installing the CT apparatus, and when the system is idle, it can be folded so as to save space effectively. This leads to a very useful technical effect.

FIG. 1 is a configuration view showing a gantry-diagnostic bed system according to a first embodiment of the present invention, which system is in an unfolding state. As shown in FIG. 1, the gantry-diagnostic bed system comprises a gantry 1 for supporting a built-in medical device, a diagnostic bed 2 for carrying an object to be inspected, a supporting lever 31, an actuating mechanism 4 and a base 51 connected to the gantry 1.

The actuating mechanism 41 is rotatably connected to the base 51 at one end and rotatably connected to the diagnostic bed 2 at the other end. The distance between the two ends can be varied via an actuating force enabling the mechanism to be telescopic. Through the base 51 and the actuating mechanism 41, the gantry 1 and the diagnostic bed 2 are formed in an integral manner. In this way, the actuating mechanism 41 can achieve a supporting function and can also enable the gantry-diagnostic bed system to be in a folding state.

The supporting lever 31 is rotatably connected to the diagnostic bed 2 at one end and detachably connected to the base 51 at the other end. The supporting lever 31 is connected to the base 51 in a detachable manner, for example, via a coupling piece such as a connecting pin or the like, such that the end of the supporting lever 31 can be conveniently detached from the base 51.

In this way, the supporting lever 31, the actuating mechanism 41 and the base 51 as a whole can be considered as a “diagnostic bed adjusting mechanism” according to the present embodiment, which may enable the diagnostic bed to move relative to the gantry and to accomplish deploying or retracting.

Now how the gantry-diagnostic bed system changes from the unfolding state to a folding state will be explained in details below.

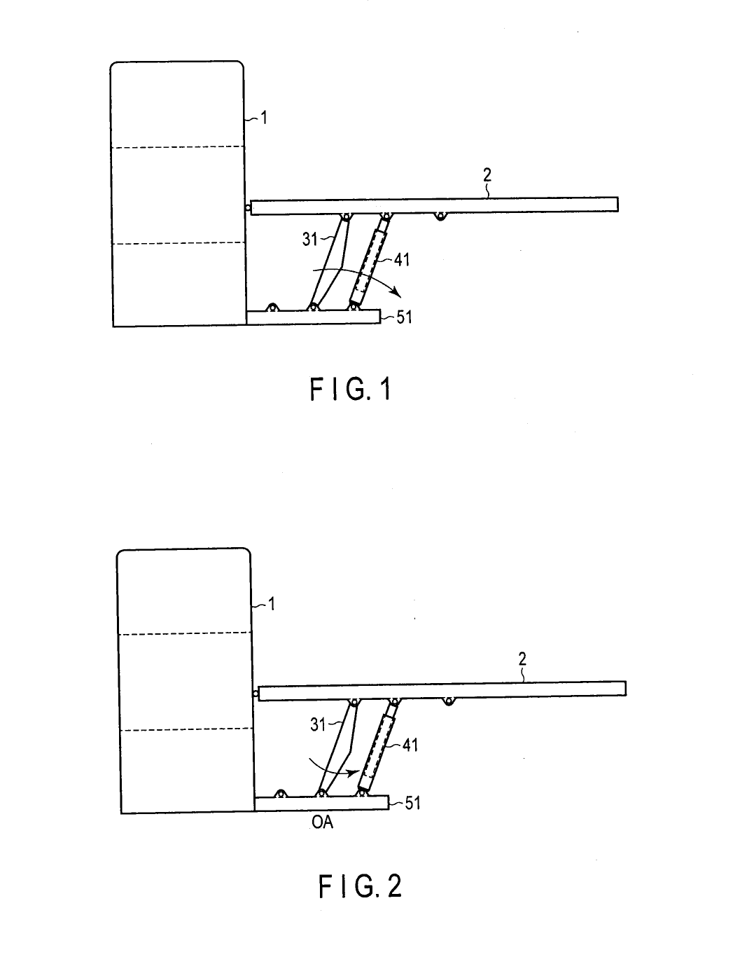

First, FIG. 2 is a configuration view showing the gantry-diagnostic bed system according to the first embodiment before being folded. As shown in FIG. 2, in an unfolding state of a normally used CT apparatus, the supporting lever 31 and the actuating mechanism 41 constitutes a parallelogram-shaped construction. Thus by rotation, the diagnostic bed can be raised or lowered.

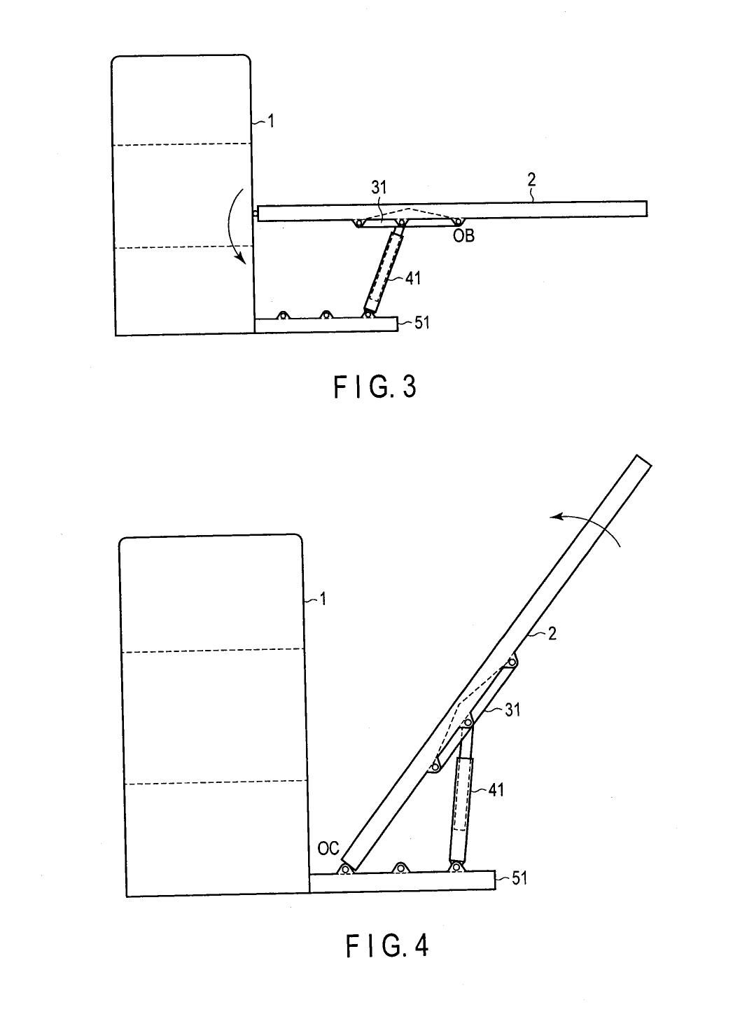

Further, when the gantry-diagnostic bed system is retracted, the supporting lever 31 is detached from the base 51 at a position OA shown in FIG. 2 (for example by pulling out a shaft pin mounted at the position) such that the supporting lever 31 is rotated in the arrow direction shown in FIG. 2 to a state shown in FIG. 3.

FIG. 3 shows a configuration view showing the gantry-diagnostic bed system according to the first embodiment during being folded. In the state shown in FIG. 3, the supporting lever 31 is locked at a position OB via an appropriate coupling piece or via suction or the like. In this way, the diagnostic bed 2 is rotatable around the actuating mechanism 41. The diagnostic bed is rotated in the arrow direction shown in FIG. 3 to a state shown in FIG. 4.

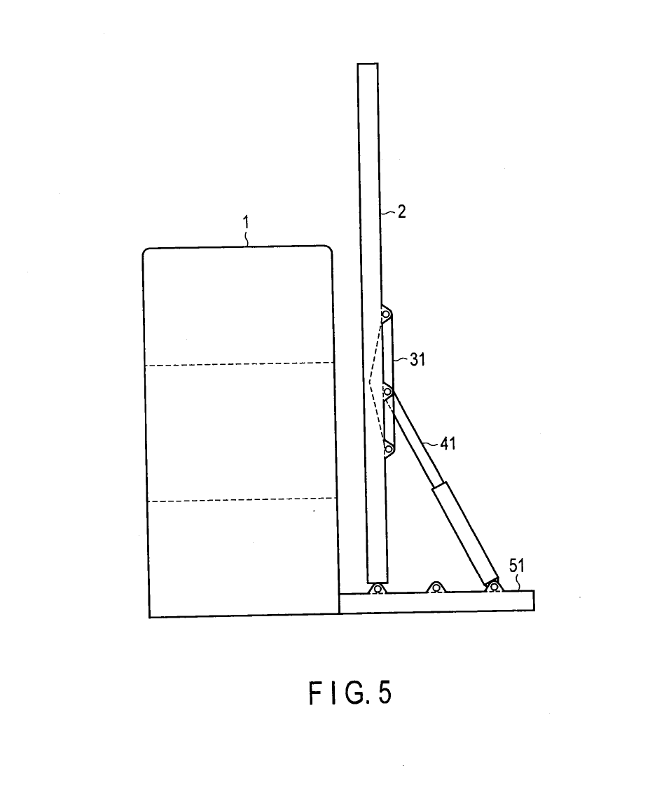

FIG. 4 is another configuration view showing the gantry-diagnostic bed system according to the first embodiment during being folded. As shown in FIG. 4, one end of the diagnostic bed 2 can be locked at a position OC for example via a shaft pin. Then, the actuating mechanism 41 accomplishes a stretching action, such that the diagnostic bed is rotated around the position OC to a state shown in FIG. 5.

FIG. 5 is a configuration view showing the gantry-diagnostic bed system according to the first embodiment, after being folded, in a compact state. As shown in FIG. 5, in a retracting state, the diagnostic bed 2 is parallel to the gantry 1. Therefore, the gantry of the integral gantry-diagnostic bed system enables to be folded relative to the diagnostic bed of the system.

A procedure that the gantry-diagnostic bed system changes from the unfolding state to the folding state is explained above. A procedure that the gantry-diagnostic bed system changes from the folding state to the unfolding state can be achieved in a reversed manner.

In the above specific embodiment, the diagnostic bed 2 is folded to be upright, i.e. to be erected perpendicularly to the floor. This may save installing space maximally. However, the present embodiment is not limited by this. It is also possible that the diagnostic bed 2 is folded obliquely relative to the gantry 2. This may also save installing space partly.

Further, the end of the diagnostic bed 2 is not limited to be secured at the position OC. It is also possible that the end of the diagnostic bed 2 is supported on the base 51 to accomplish folding. In this case, the base can be provided with a corresponding location groove.

### Second Embodiment

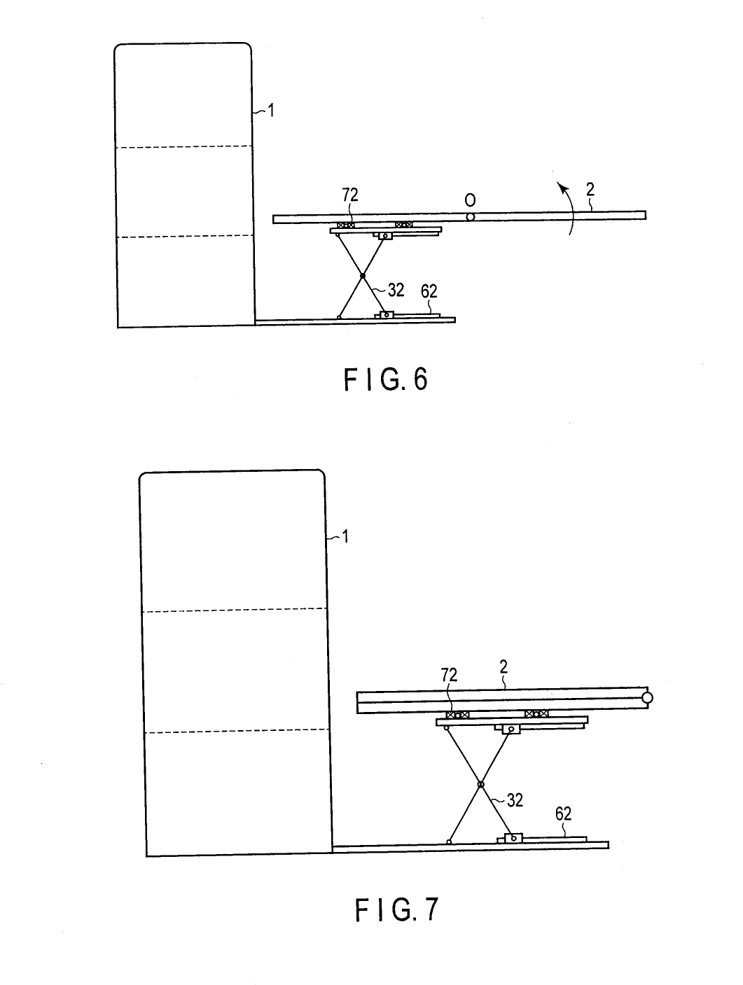

Now a second embodiment of the present invention will be explained with respect to FIGS. 6 to 9, wherein the same parts as those of the first embodiment are expressed by the same reference numerals.

FIG. 6 is a configuration view showing a gantry-diagnostic bed system according to the second embodiment of the present invention, which system is in an unfolding state. As shown in FIG. 6, the gantry-diagnostic bed system comprises a gantry 1 for supporting a built-in medical device, a diagnostic bed 2 for carrying an object to be inspected, an X-formed connecting part 32, a linear guide 62 and a turntable bearing 72.

The X-formed connecting part 32 and the linear guide 62 can be considered as an integral “diagnostic bed lifting mechanism”. Specifically, the X-formed connecting part 32 and the linear guide 62 are matched with each other to constitute a construction for coupling the gantry 1 with the diagnostic bed 2 in an integral manner and for supporting the diagnostic bed 2. As shown in FIG. 6, both ends of one side of the X-formed connecting part 32 are installed such that they are not slidable relative to the linear guide 62, and both ends of the other side of the X-formed connecting part are slidably connected on the linear guide 62. Further, the lower guiding part of the linear guide 62 is integrally connected with the gantry 1.

The upper guiding part of the linear guide supports the turntable bearing 72. The diagnostic bed 2 is carried on the turntable bearing 72 such that the diagnostic bed 2 is rotatable in the horizontal plane.

By this kind of structure that enables the X-formed connecting part 32 to match with the linear guide 62, both ends of one side of the X-formed connecting part 32 are slidable on the guide such that the diagnostic bed 2 can be raised or lowered vertically.

Further, the diagnostic bed 2 can have a hinge O along its length direction to divide the diagnostic bed 2 into two portions, such that one portion of the diagnostic bed 2 not carrying by the turntable bearing 72 can be folded upwardly and can be overlapped against the other portion of the diagnostic bed. FIG. 7 shows how the diagnostic bed 2 having the hinge O is overlapped. Since the diagnostic bed 2 is overlapped along the length direction as show in FIG. 7, the diagnostic bed 2 can be shorted in its length so as to facilitate retracting the diagnostic bed.

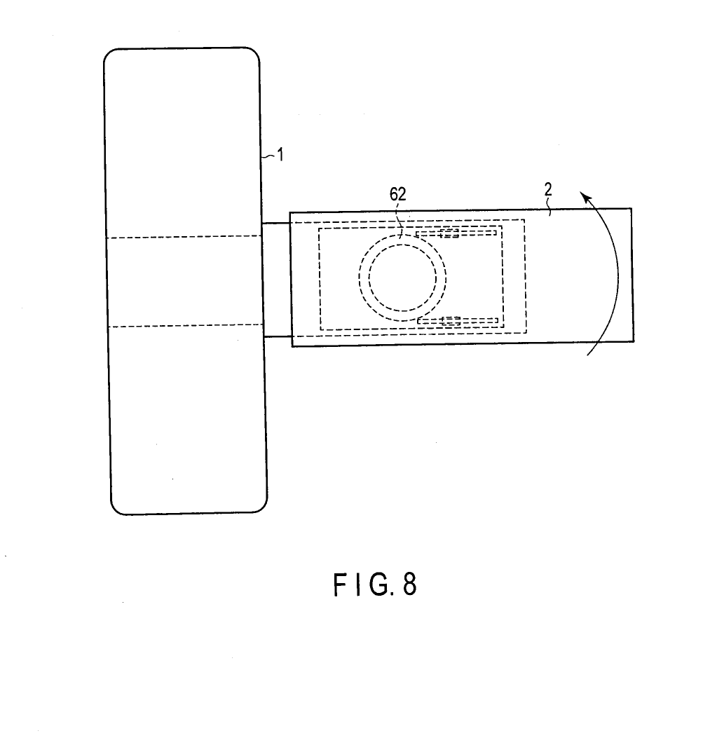

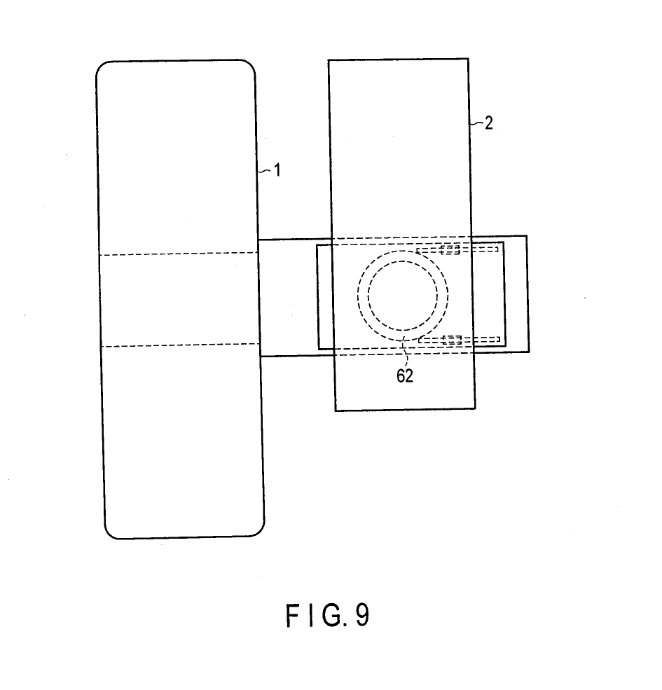

It is also feasible that the diagnostic bed 2 does not have said hinge O such that it is unnecessary to overlap the diagnostic bed 2 as shown in FIG. 7 upon folding. Now a procedure that the gantry-diagnostic bed system, in which the diagnostic bed 2 has no hinge O, changes from the unfolding state to a folding state will be explained with respect to FIGS. 8 and 9.

First, FIG. 8 is a top view showing the gantry-diagnostic bed system according to the second embodiment in the unfolding state. As shown in FIG. 8, in the unfolding state of the normally used CT apparatus, the diagnostic bed 2 can be rotated along the arrow direction in the horizontal plane via the turntable bearing 72. The diagnostic bed 2 is rotated by 90 degrees to a state shown in FIG. 9.

FIG. 9 is a top view showing the gantry-diagnostic bed system according to the second embodiment in the folding state. In the folding state, the diagnostic bed 2 is parallel with the gantry 1 such that the gantry-diagnostic bed system occupies a shorter space along a direction perpendicular to the gantry 1. In this way, by simple rotation, the integral gantry-diagnostic bed system can be folded.

The procedure that the gantry-diagnostic bed system changes from the unfolding state to a folding state has been explained above. A procedure that the gantry-diagnostic bed system changes from the folding state to the unfolding state can be achieved in a reversed manner.

In the above embodiment, using the structure that enables the X-formed connecting part 32 to match with the linear guide 62, the diagnostic bed 2 can be raised or lowered. However, the present embodiment is not limited by this. It is also possible that any appropriate known structure enabling the diagnostic bed 2 to be lifted can be used as a mount for supporting the turntable bearing 72. Any appropriate structure enabling the turntable bearing 72 to connect with the diagnostic bed 2 is feasible.

Further, in the above specific embodiment, the diagnostic bed 2 in the folding state is rotated by 90 degrees to a state in which the diagnostic bed is parallel with the gantry 1, such that space is saved maximally. However, the present embodiment is not limited by this. It is also possible that the diagnostic bed 2 is rotated by an angle of less than 90 degrees such that the diagnostic bed 2 is arranged obliquely relative to the gantry 1. In this way, space can be saved partly.

Further, the diagnostic bed 2 can be provided with more than one hinge O, such that the diagnostic bed 2 can be overlapped in a multi-layer manner.

### Third Embodiment

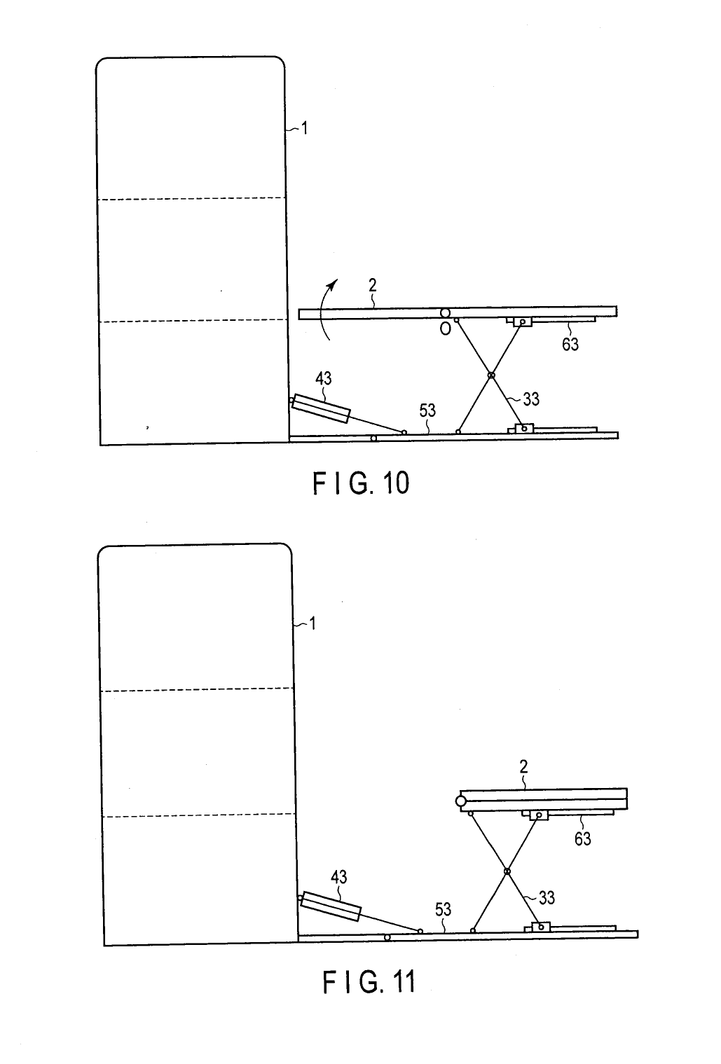

Now a third embodiment of the present invention will be explained with respect to FIGS. 10 to 13, wherein the same parts as those of the first embodiment are expressed by the same reference numerals.

FIG. 10 is a configuration view showing a gantry-diagnostic bed system according to the third embodiment of the present invention in an unfolding state. As shown in FIG. 10, the gantry-diagnostic bed system comprises a gantry 1 for supporting a built-in medical device, a diagnostic bed 2 for carrying an object to be inspected, an actuating mechanism 43, a base 53, an X-formed connecting part 33 and a linear guide 63.

The diagnostic bed 2 has a hinge O along its length direction such that the diagnostic bed 2 can be overlapped itself.

Further, in this embodiment, the base 53 is connected to the gantry 1 and also has a hinge along its length direction, such that the base 53 is divided into two portions, of which one portion is adjacent the gantry 1 and the other portion is far away from the gantry via the hinge.

The actuating mechanism 43 is connected to both the gantry and the portion, far away from the gantry via the hinge, of the base.

The X-formed connecting part and the linear guide can be considered as a “diagnostic bed lifting mechanism”. The diagnostic bed is supported on the portion, far away from the gantry via the hinge, of the base such that the diagnostic bed can be raised or lowered. Specifically, as show in FIG. 10, the lower guiding part of the linear guide 63 is formed integrally with the base. Both ends of one side of the X-formed connecting part 33 are mounted on the diagnostic bed 2 and the base 53 respectively such that the ends are not slidable, and both ends of the other side of the X-formed connecting part are slidably connected on the linear guide 63.

Using this kind of structure that enables the X-formed connecting part 33 to match with the linear guide 63, both ends of a corresponding side of the X-formed connecting part 33 are slidable on the guide such that the diagnostic bed 2 can be raised or lowered vertically.

In the third embodiment, using the structure that enables the X-formed connecting part 33 to match with the linear guide 63, the diagnostic bed 2 can be raised or lowered. However, the present embodiment is not limited by this. It is also possible that any appropriate known structure enabling the diagnostic bed 2 to be lifted can be adopted. That is, any appropriate structure enabling the diagnostic bed 2 to be lifted is feasible.

Further, the diagnostic bed 2 has a hinge O along its length direction such that the diagnostic bed 2 is divided into two portions, of which one portion is adjacent the gantry 1 and the other portion is far away from the gantry 1. In this way, the diagnostic bed 2 can be overlapped itself on the X-formed connecting part 33 such that the diagnostic bed 2 can be shorted along its length direction. FIG. 11 shows how the diagnostic bed 2 having the hinge is overlapped. As shown in FIG. 11, the X-formed connecting part 33 is connected to the portion of the diagnostic bed 2 far away from the gantry 1. By folding the portion of the diagnostic bed 2 adjacent the gantry 1 upwardly, this portion is overlapped against the portion of the diagnostic bed far away from the gantry 1.

A procedure that the gantry-diagnostic bed system according to the third embodiment of the present invention changes from the unfolding sate to a folding state is explained below.

First, as shown in FIG. 11, the two portions, which are divided by the hinge O, of the diagnostic bed 2 are overlapped one on the other.

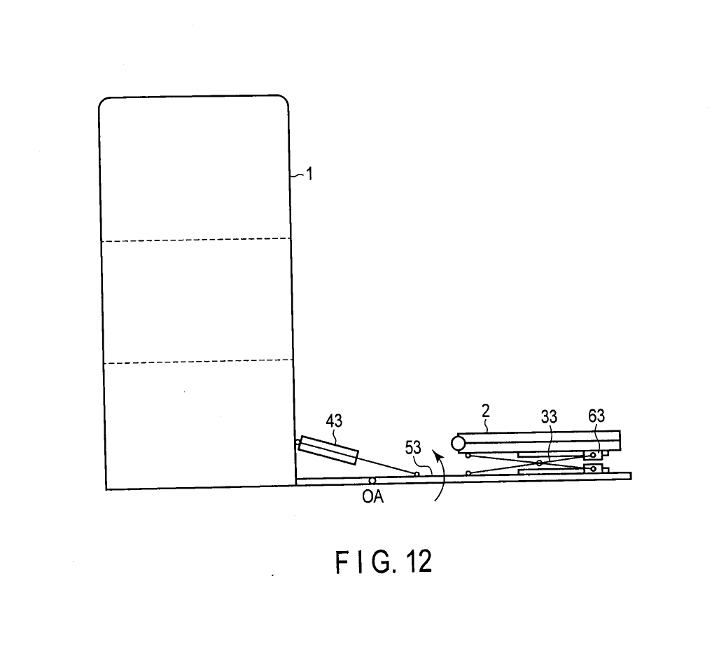

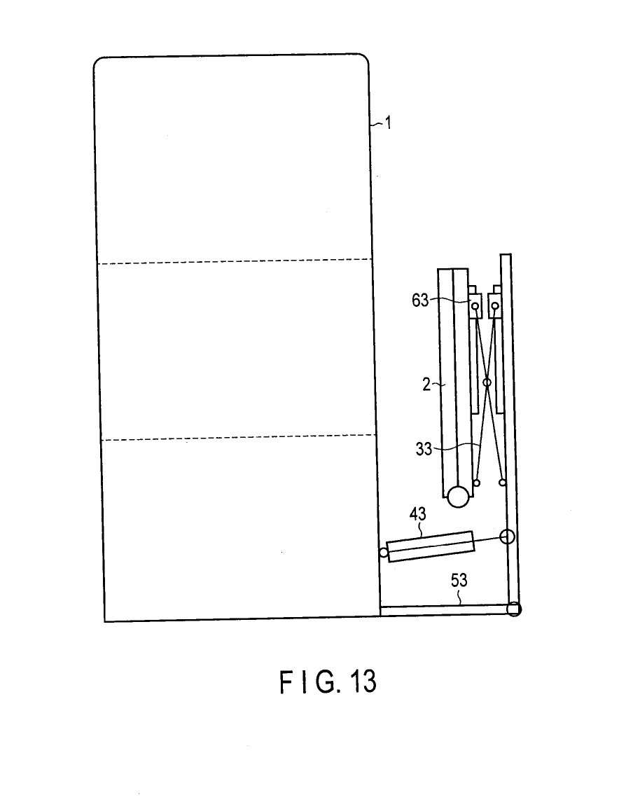

Then, as shown in FIG. 12, the diagnostic bed 2 is lowered using the X-formed connecting part 33 and the linear guide 63. The actuating mechanism is contracted such that the portion, far away from the gantry via the hinge, of the base 53 is rotated around the hinge OA in the arrow direction shown in FIG. 12 by 90 degrees. Finally, this portion is rotated to a state shown in FIG. 13.

FIG. 13 is a configuration view showing the folded gantry-diagnostic bed system according to the third embodiment in a compact state. As shown in FIG. 13, the portion, far away from the gantry via the hinge, of the base 53 is pulled upright such that this portion is parallel with the gantry 1. The retracted diagnostic bed 2 is received above the portion of the base 53 adjacent the gantry. Therefore, the gantry-diagnostic bed system is further folded and retracted.

The procedure that the gantry-diagnostic bed system changes from the unfolding state to a folding state has been explained above. A procedure that the gantry-diagnostic bed system changes from the folding state to the unfolding state can be achieved in a reversed manner.

Further, in the above specific embodiment, the portion, far away from the gantry via the hinge, of the base 53 is parallel with the gantry 1 in the folding state such that space is saved maximally. However, the present embodiment is not limited by this. It is also possible that the portion, far away from the gantry via the hinge, of the base 53 can be oblique relative to the gantry 1 in the folding state such that space can also be saved partly. Further, the diagnostic bed 2 can be provided with more than one hinge O, such that the diagnostic bed 2 can be overlapped in a multi-layer manner.

According to the embodiments of the present invention explained above, the gantry and the diagnostic bed are designed in an integral manner such that the gantry-diagnostic bed system in the folding state is packed and transported one time. Thus the number and volume of the package can be reduced.

Further, after the integral gantry-diagnostic bed system is transported into the hospital, it is unnecessary to adjust the positional relationship between the gantry and the diagnostic bed. It is only necessary to release the gantry-diagnostic bed system from the folding state and then to secure the gantry. Thus it saves time for maintainers and doctors to adjust the system.

Further, in the case that the CT apparatus is not operated, the diagnostic bed can be folded to save the space of the hospital.

Further, if it is required to modify the position of the CT apparatus due to some reason, it is just necessary to move the system in the folding as a whole. In comparison with the conventional system in which the gantry and the diagnostic bed are separated from each other, the inventive system can save considerable time and facilitate the operation of operators.

Therefore, the present invention is adapted for several technical fields in which medical devices for diagnosing an object to be inspected on a diagnostic bed are retracted using a gantry.

While certain embodiments have been described, these embodiments have been presented by way of example only, and are not intended to limit the scope of the inventions. Indeed, the novel embodiments described herein may be embodied in a variety of other forms; furthermore, various omissions, substitutions and changes in the form of the embodiments described herein may be made without departing from the spirit of the inventions. The accompanying claims and their equivalents are intended to cover such forms or modifications as would fall within the scope and spirit of the inventions.

What is claimed is:

1. A gantry-diagnostic bed system comprising:
a gantry for supporting a medical device;
a diagnostic bed designed as being integral with the gantry and for carrying an object to be inspected; and
a diagnostic bed adjusting mechanism for adjusting the position of the diagnostic bed relative to the gantry such that the diagnostic bed can be folded with respect to the gantry.

2. The gantry-diagnostic bed system according to claim 1, wherein the diagnostic bed adjusting mechanism can be used to adjust the position of the diagnostic bed relative to the gantry such that the diagnostic bed can be upright and thus be parallel with the gantry.

3. The gantry-diagnostic bed system according to claim 1, wherein the diagnostic bed has a hinge along its length direction such that the diagnostic bed can be self-folded to yield overlapping.

4. The gantry-diagnostic bed system according to claim 1, wherein the diagnostic bed adjusting mechanism comprises:
a base connected to the gantry, the base carrying an actuating mechanism and a supporting lever;
the actuating mechanism for supporting the diagnostic bed and providing a folding force for the diagnostic bed; and
the supporting lever which can be switchable between first and second positions, wherein in the first position, the supporting lever is arranged parallel with the actuating mechanism and cooperates with it to support the diagnostic bed in the horizontal direction, and in the second position, the supporting lever is retreated in such a way that it is parallel with the diagnostic bed,
wherein when the supporting lever is in the second position, the actuating mechanism provides the folding force such that the diagnostic bed can be folded with respect to the gantry.

5. The gantry-diagnostic bed system according to claim 1, wherein the diagnostic bed adjusting mechanism comprises a diagnostic bed lifting mechanism and a turntable bearing, as a rotational component, which is rotatable about the diagnostic bed lifting mechanism, wherein the diagnostic bed lifting mechanism is secured on the gantry, and the rotational component is secured on the diagnostic bed such that the diagnostic bed is rotatable in the horizontal plane and thus is foldable with respect to the gantry.

6. The gantry-diagnostic bed system according to claim 5, wherein the diagnostic bed lifting mechanism comprises an X-formed connecting part and a linear guide, both ends of one side of the X-formed connecting part are slidably connected to the horizontal linear guide respectively to support the rotational component.

7. The gantry-diagnostic bed system according to claim 1, wherein the diagnostic bed has a hinge along its length direction, and the diagnostic bed adjusting mechanism includes:
a base connected to the gantry and having a hinge along its length direction;
an actuating mechanism which connects the gantry with a portion, far away from the gantry via the hinge, of the base; and
a diagnostic bed lifting mechanism which supports the diagnostic bed on the portion, far away from the gantry via the hinge, of the base and lifts the diagnostic bed.

8. The gantry-diagnostic bed system according to claim 7, wherein the diagnostic bed lifting mechanism comprises an X-formed connecting part and a linear guide, wherein both ends of one side of the X-formed connecting part are slidably connected to the horizontal linear guide respectively.

9. A method for folding a gantry-diagnostic bed system according to claim 4, comprising:
a step of enabling the supporting lever to be in the second position; and
a step of driving the actuating mechanism such that the diagnostic bed is upright with respect to the gantry.

10. A method for folding a gantry-diagnostic bed system according to claim 5, comprising:
a step of rotating the rotational component with respect to the gantry-diagnostic bed lifting mechanism such that the diagnostic bed is rotated in the horizontal plane to a state in which it is parallel with the gantry.

11. A method for folding a gantry-diagnostic bed system according to claim 7, comprising:
a step of overlapping the diagnostic bed via the hinge on the diagnostic bed lifting mechanism;
a step of lowering the diagnostic bed be means of the diagnostic bed lifting mechanism; and
a step of pulling up the portion, far away from the gantry via the hinge, of the base by means of the actuating mechanism.
